(19) United States
(12) Patent Application Publication
(10) Pub. No.: US 2003/0143746 A1
(43) Pub. Date: Jul. 31, 2003

(54) Self-calibrating body anayte monitoring system

(76) Inventor: Burton H. Sage JR., Vista, CA (US)

Correspondence Address: BURTON H. SAGE, Jr., 3430 BERNARDINO LANE, VISTA, CA, 92084, US

(21) Appl. No.: 10/059,390

(22) Filed: Jan. 31, 2002

(51) Int. Cl. G01N 31/00; A61B 5/00

(52) U.S. Cl. 436/8; 436/14; 435/14; 600/309; 600/365; 600/573; 600/576; 600/583; 73/1.01; 73/1.02

(57) ABSTRACT

A wearable periodic self-calibrating body analyte monitoring system based on the principles of microdialysis for measurement of a body analyte is disclosed. In a preferred embodiment, the system is designed to measure glucose, and can be held on the body with a skin adhesive for comfort. The system may be combined with an insulin delivery system to create an artificial pancreas.

### BACKGROUND OF THE INVENTION

A. Field of the invention. The present invention relates in general to medical devices. Specifically, the invention relates to devices and methods for measuring the concentration of therapeutically useful compounds in body fluids.

B. Related Art. Microdialysis systems intended to measure the concentration of a body analyte, including systems to measure glucose, are known. In 1987 Lonnroth, et al published “A microdialysis method allowing characterization of intercellular water space in humans” in the American Journal of Physiology 253:E228-E231. Further, in 1995, Stemberg, et al published “Subcutaneous glucose in humans: real time estimation and continuous monitoring” in Diabetes Care 18:1266-1269.

The purpose of these efforts and devices, and the efforts and devices of many others, was to improve the methods of measuring glucose in blood and other body fluids, and thereby improve the quality of therapy for diabetes. In spite of these efforts, while significant progress has been made, there is yet no basis for a suitable product based on microdialysis.

Many products are currently marketed to measure blood glucose. One class of these products, known as glucose strips and meters, require a blood sample, usually from a fingertip. They provide a satisfactory result when they are used, but they only provide a single result for each use. In diabetes, the glucose concentration in the body can change so quickly and so much that a single measurement, while being meaningful at the time it is taken, has little value a short time later. In general, the more frequently the glucose concentration is measured, the better diabetes can be managed. From a practical point of view, though, a new and accurate glucose measurement with minimal time lag (delay caused by the time it takes to remove the specimen and make the measurement) every three to five minutes is adequate to effectively manage even the most brittle cases of diabetes.

This need for more frequent glucose measurements led to a second class of glucose measuring systems (known as “needle” sensors) that monitor glucose continuously. For over two decades, devices of this class, that measure glucose in a blood vessel or in interstitial fluid just below the surface of the skin, have been under development. Recently, such a device for use in interstitial fluid, developed by the MiniMed Corporation, was approved for sale. It can be used for up to three days.

This product, and other “needle sensors” currently under development, must be calibrated by a blood glucose measurement, usually obtained from fingerstick blood using a “strip and meter” device. The need for calibration is caused by a decrease in the sensitivity of the sensor to glucose over time during use. The sensor must be calibrated once when the product is first placed in the skin and, in the case of the approved product, as frequently as every eight hours until it is removed. While this system does provide superior glucose information, it is much more inconvenient for the user, who must both insert the needle and provide calibration as needed from fingerstick glucose measurements.

To avoid the decrease in sensitivity with time exhibited by the “needle sensors’, microdialysis systems for glucose were developed. These systems moved the actual glucose detector from the tip of the needle sensor, which is inside the body, to a place outside the body. This change of location resulted in a much more stable glucose sensitivity. However, a microdialysis system is more complicated than a needle sensor, and early versions required perfusion of large volumes of fluid through the microdialysis needle, making the device too big for routine personal use. The volumes of fluids required for a day of use, for example, in the microdialysis system described by Pfeiffer in U.S. Pat. No. 5,640,954, were measured in hundreds of milliliters to liters per day.

Korf, in U.S. Pat. No. 6,013,029 describes an improved microdialysis system that uses much less fluid. In the preferred flow rate range specified by Korf, less than 20 microliters per hour, the amount of fluid required for a day's use is less than 480 microliters, a volume that can be very comfortably worn.

As advanced as Korf's system is, though, it still suffers from at least three problems. First, the flow through the system is continuous. Constant continuous flow of fluid, especially at the very slow flow rates described by Korf, is hard to establish and maintain. For example, the very low flow rates imply that the flow is driven by very low pressure differentials and driving forces. Thus even modest changes in atmospheric pressure, from weather systems or from traveling from Los Angeles to Denver, can result in significant flow rate changes. Also, for each of the fluid driving means described by Korf, as time passes, the flow rate will decrease. This happens as the fluid absorbing material is consumed, or due to backpressure developed in the capillary or behind the osmotic membrane, or through filling of the pressure differential reservoir. Korf makes no provision to compensate for this flow rate change.

Second, a constant perfusate flow rate requires the body analyte to be measured by a sensor that measures the analyte by the rate at which a reaction occurs which in turn depends on the concentration of the analyte to be measured in the perfusate (as opposed to a sensor that measures the quantity of the analyte in a volume). Korf makes reference to an amperometric sensor that is sensitive to the concentration of hydrogen peroxide (or oxygen) present in the perfusate. These rate sensors are, by their nature, noisier and less accurate than a sensor that measures the total quantity of analyte present.

Third, Korf makes no provision for calibration of his system. At the very least, manufacturing variations will require that each system be calibrated before use. Also, no provision is made to accommodate variations in the degree of equilibrium achieved between the glucose concentration in the perfusate and the glucose concentration in the interstitial fluid. This degree of equilibrium is commonly referred to as yield. Yield varies directly with flow rate, implying the need for recalibration over time as the driving force is reduced. Further, flow rate changes due to changes in atmospheric conditions, or travel, or other system changes may require additional calibrations.

Thus, while the system disclosed by Korf provides significant improvements over other older and larger microdialysis systems by dramatically reducing the volume of fluids, there is still room for improvement.

Pfeiffer, in U.S. Pat. No. 6,091,976, provides for non-continuous flow of the perfusate during a portion of the time of operation of the system to decrease the average flow rate to increase the yield of glucose during this time. Further, glucose is added to the perfusate to avoid “impoverishment” of the analyte in the tissue, and to provide a system calibration during a second high flow rate period of operation. However, this method places high demand on the accuracy of the assay, since the concentration of the analyte in the tissue during the low flow rate portion of operation now must be calculated from the difference between the concentration of glucose added to the perfusate and the concentration of glucose measured in the perfusate after microdialysis. And when the assay is an enzyme catalyzed reaction, which is known to be subject to drift and temperature variations, the accuracy problem can be especially acute.

Further, the glucose containing perfusate that passes through the microdialysis needle during the high flow rate portion of operation will lose glucose to or gain glucose from the tissue depending on the tissue concentration, thereby altering the concentration of the glucose in the perfusate. Hence the accuracy of the “calibration” glucose concentration is questionable as well. In U.S. Pat. No. 6,091,976 Pfeiffer in principle improves the art by providing means to improve yield and calibrate the sensor. In fact, though, the specific means disclosed introduce inaccuracies of their own.

As can be seen from the issues and problems arising from prior art methods, there still remains a need for accurate, reliable, and convenient methods and systems to provide frequent measurement of body analytes.

### BRIEF SUMMARY OF THE INVENTION

It is an object of this invention to provide a body analyte monitoring system with a self-calibration means so that the system may be used without the user obtaining and entering a calibration measurement at any time during its use. Accordingly, a calibration fluid containing reservoir and means to cause this calibration fluid to react with an appropriate reagent and flow to an analysis chamber are provided. As is described in detail in the next paragraphs, an assay conducted on this calibration fluid is alternated with an assay conducted on a body analyte laden perfusate to provide frequent calibration of the assay of the body analyte laden perfusate.

It is a further object of this invention to provide a body analyte monitoring system that provides a steady stream of frequent, accurate, and discrete measurements of the concentration of a body analyte, in particular glucose. The word periodic is used herein to mean a steady stream of discrete measurements, and to distinguish the body analyte monitoring system of this invention from continuous body analyte monitoring systems known in the art. Accordingly, in a preferred embodiment, a microdialysis needle with a very low internal volume and shallow cross-sectional aspect ratio of height to width is provided. Further, perfusate is caused to flow through the microdialysis needle at a sufficiently low flow rate that the time for the body analyte to diffuse into the lumen of the microdialysis needle and reach a concentration equilibrium with the body analyte in the body tissue is shorter than the transit time of the microdialysis fluid through the microdialysis needle. Thus impoverishment of the interstitial fluid of the analyte is avoided and the yield of body analyte captured by the perfusate is nearly 100%. The flow of the body analyte enriched perfusate from the microneedle continues to a junction where it is merged with a solution containing a reagent specific for the body analyte. The reagent may be an enzyme such that the subsequent assay is electrochemical for products of the reaction of the enzyme with the body analyte, or the reagent may be a viscosity altering compound such that the subsequent assay measures the change in solution viscosity caused by the reaction of the body analyte with the viscosity altering compound, or the reagent may be a compound that alters the optical properties of the solution such that the subsequent assay measures the change in an optical property of the solution caused by the reaction between the body analyte and the optical property altering compound.

The merged perfusate and reagent solutions flow to an analysis chamber that has an analysis volume larger than the internal volume of the microdialysis needle. At selected times, the flow of the mixed perfusate and reagent solution is stopped so that an assay for the body analyte may be conducted in the assay chamber. Stopping the flow allows the reaction within the assay chamber to be continued until the reacting species are exhausted, or until the measurement of the solution viscosity or the measurement of the optical property has stabilized, thereby avoiding reaction kinetics issues such as temperature and sensitivity. Finally, in this preferred embodiment, the flow of the perfusate from the microdialysis needle to the junction is alternated with flow of a calibration fluid to the junction. In this way, the reagent solution alternately mixes with the sample-laden perfusate from the microdialysis needle and the calibration fluid, providing a calibration of the assay. In the case of an enzyme reaction, since the reaction is carried out to completion, there is no contamination between calibration assay and perfusate assay.

It is a further object of the invention to provide a body analyte monitoring system that minimizes the volume of reagents required to carry out the measurement of the concentration of a body analyte. In a preferred embodiment of the invention, where a new measurement is obtained every 5 minutes, the perfusate flows through the microdialysis needle for a period of 45 seconds at a flow rate of 1 nanoliter per second. The total volume of fluids, including perfusate, enzyme solution and calibration fluid, required to operate the system for three days is less than 250 microliters.

It is a further object of the invention to provide a body analyte monitoring system that minimizes the lag time, that is, the time required to obtain the sample and perform the assay of the concentration of a body analyte. In a preferred embodiment of the invention, the lag time is one minute.

It is a yet another object of the invention to provide a body analyte monitoring system that minimizes the size of the system so that it may be comfortably worn. Accordingly, in a preferred embodiment of the invention, the fluid driving means for the perfusate, reagent, and calibration fluids is a pressurized fluid reservoir system. This eliminates the need for rotating electrical machinery such as a pump and simultaneously reduces the size of the battery since power to drive the pump isn't needed.

It is a further object of the invention to minimize the discomfort of needle insertion required for access of the body fluid containing the body analyte. In a preferred embodiment of the invention, access to the body analyte containing tissue fluid, for example interstitial fluid, is obtained with a microfabricated microdialysis needle 5 mm long and 150 microns wide by 100 microns thick. The fabrication of such a microneedle is described in “An integrated microfluidic device for the continuous sampling and analysis of Biological Fluids” by Zahn, Jeffrey D., et al in the Proceedings of the ASME IMECE MEMS 2001 Symposium, New York, N.Y., November 2001, incorporated herein by reference. Other methods of fabrication of dual lumen microneedles of this size are also known in the art, especially the HexSil method of silicon micromolding developed at the University of California, Berkeley by Pisano, Albert A, Evans, John, and Talbot, Nick.

It is a yet further object of the invention to provide a means to measure glucose accurately and sufficiently often to control the administration of insulin, thereby controlling the glucose level in the body. Accordingly, an insulin administration device may be combined with the body analyte monitoring device, in this case a glucose monitoring device, and the glucose measurements may be used to control the rate of insulin administration, thereby creating an artificial pancreas.

### BRIEF DESCRIPTION OF THE DRAWINGS

FIG. 1 is a schematic of a preferred embodiment of the body analyte monitoring system.

FIG. 2 is plan view of the microdialysis needle and microfluidics chip of a preferred embodiment of the body analyte monitoring system.

FIG. 3 is the cross sectional view of the microdialysis needle of a preferred embodiment of the body analyte monitoring system.

FIG. 4 is a schematic of the fluid sequencing subsystem for the perfusate, the reagent, and the calibration fluids of a preferred embodiment of the body analyte monitoring system.

### DETAILED DESCRIPTION OF THE INVENTION

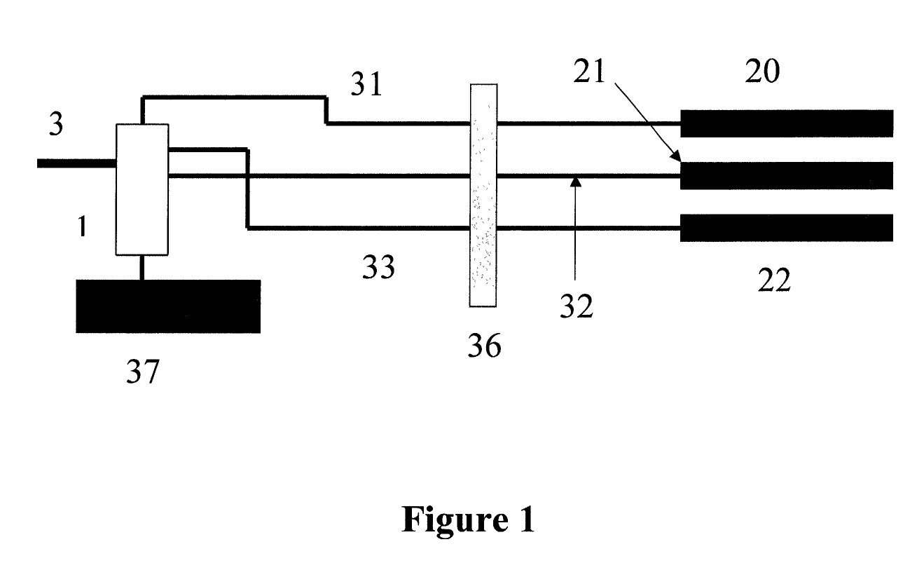

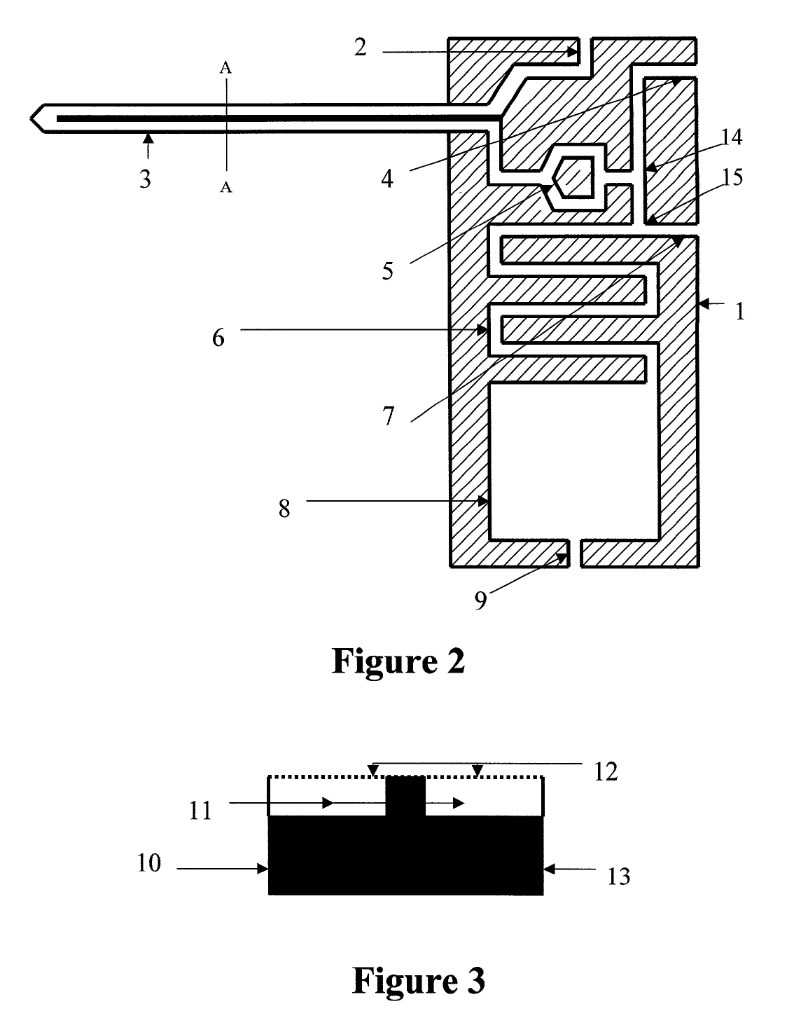

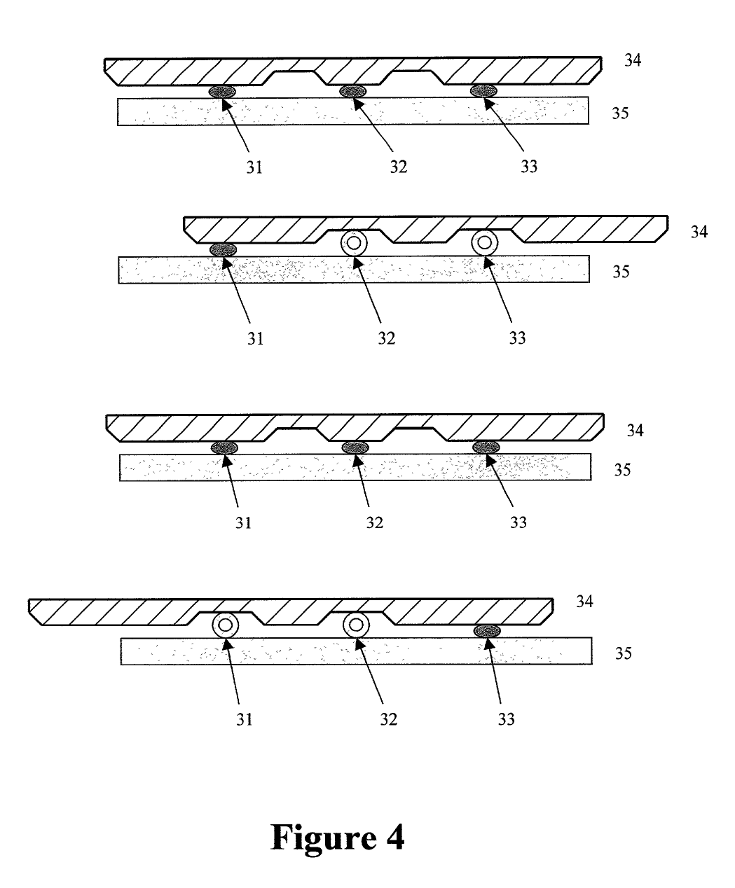

A schematic of an apparatus for obtaining periodic, self-calibrated measurements of a body analyte is shown in FIG. 1. The apparatus is comprised of microfluidics chip 1 which is attached, either integrally or by a fluid connecting means, to microdialysis needle 3. Microfluidics chip 1, shown in greater detail in FIG. 2, is supplied with perfusate through fluid supply line 31 from perfusate reservoir 20, with enzyme solution through fluid supply line 32 from enzyme solution reservoir 21, and with calibration fluid through fluid supply line 33 from calibration fluid reservoir 22. The perfusate is preferably an isotonic solution composed of saline and containing other compounds to make the fluid biocompatible. The enzyme solution is also preferably an isotonic solution of saline, but it also contains an enzyme specific for the body analyte of interest. If the body analyte is glucose, then the enzyme is preferably glucose oxidase. The calibration fluid is also preferably an isotonic solution of saline, but it also contains a known concentration of the body analyte of interest. Preferably, the perfusate, enzyme solution, and calibration fluids also contain stabilizers or preservatives as needed to insure that these fluids are stable during their shelf life. Fluid supply lines 31, 32, and 33 are made of any of a number of flexible tubing materials such as Tygon and silicone rubber. Fluid containing reservoirs 20, 21, and 22 are made of any of a number of laminated films composed of a fluid compatible fluid contacting inner layer of, for example, polyethylene, and a gas and vapor impermeable layer such as aluminum. Other layers in the laminate may be, as needed, a material such as PET for tensile strength and a light absorbing layer for radiation protection. Fluid is caused to flow from these reservoirs to the analysis chamber by pumping means such as one or more positive displacement pumps, but preferably by pressure applied to the reservoirs by constant pressure springs (not shown—for example, the springs described in Sage, et. al. in U.S. Pat. No. 5,957,895). These three fluids, the perfusate, the enzyme containing fluid, and the calibration solution, are sequenced into microfluidics chip 1 by means of fluid sequencing subsystem 36, shown in greater detail in FIG. 4. All fluids pass through microfluidics chip 1 and are collected in waste container 37.

In order to collect a sample of the body fluid, microdialysis needle 3 is placed in a body fluid, preferably interstitial fluid just below the surface of skin. As shown in FIG. 2, perfusate flows into microdialysis chip 1 through perfusate entry 2, and flows the entire length of microdialysis needle 3 from the end proximal to microdialysis chip 1 to its distal end and back to the proximal end before passing back into microdialysis chip 1 and through check valve 5. As the perfusate passes through microdialysis needle 3, body analyte enters the perfusate by diffusion through a semipermeable membrane 12, shown in greater detail in FIG. 3, which shows a cross section of microdialysis needle 3. In a preferred embodiment of the invention, the flow rate through microdialysis needle 3 is 1 nanoliter per second, and the dimensions of the lumen 11 of microdialysis needle 3 are 20 microns high by 50 microns wide. The semipermeable region of microdialysis needle 3 is 5 millimeters long, making the region of microdialysis 10 millimeters in length. Thus the transit time of fluid entering the microdialysis needle 3 is ten seconds. Given the rapid diffusion of low molecular body analytes such as lactate and glucose (the diffusion constant for glucose in a low viscosity fluid such as water is 6.7×10⁻⁶ cm²/sec) and the relatively shallow lumen of microdialysis needle 3, diffusion equilibrium for the analyte is rapidly reached between the interstitial fluid and the perfusate. In the preferred embodiment described here, the equilibrium time is 0.64 seconds (diffusion time is calculated using the equation t=x²D where t is the diffusion time, x is the diffusion distance, in this case the height of the lumen of the microdialysis needle, and D is the diffusion constant). In this preferred embodiment a high yield of the body analyte in the perfusate is provided and reduction of the concentration of the body analyte in the tissue adjacent the microdialysis needle is avoided. The microdialysis needle of the preferred embodiment can be made using microfabrication techniques as described in Zahn, et al (above) using several different materials. Preferred materials are silicon or quartz for biocompatibility. If the microdialysis needle and microdialysis chip are made separately and joined during manufacture, the microdialysis chip can also be made by molding or embossing using a variety of polymers including polycarbonate and polyethylene.

In the sequence of operation of this microdialysis system, while perfusate is flowing, calibration fluid is not flowing (as shown in the bottom illustration in FIG. 4, which shows the perfusate supply line and the enzyme supply line open, but the calibration fluid supply line closed). Thus at junction 14, the body analyte laden perfusate moves to junction 15 where it is joined with enzyme solution entering the microdialysis chip at entry 7. As the perfusate and enzyme solution travel the length of mixing channel 6, the analyte, for example, glucose, and the enzyme, for example glucose oxidase, diffuse together and react, creating the reaction components hydrogen peroxide and gluconic acid (the details of this reaction are described in detail in U.S. Pat. No. 5,640,954, which is incorporated herein by reference). In a preferred embodiment of the invention, the mixing channel 6 is 10 mm long with lumen dimensions of 20 microns in high and 50 microns wide. The combined flow rate of the perfusate and the enzyme solution in the preferred embodiment is 2 nanoliters per second and the dwell time in the mixing channel for the combined perfusate and enzyme solution is 5 seconds. The time for the glucose to mix into the enzyme solution in this preferred embodiment, assuming laminar flow in the mixing channel and diffusion of the glucose into the enzyme solution, is 0.9 seconds, well shorter than the dwell time.

The mixed and reacted perfusate and enzyme solution proceed from mixing channel 6 to analysis chamber 8. The concentration of the reaction products of the enzyme and body analyte, which is in direct proportion with the concentration of the body analyte in the perfusate exiting the microdialysis needle, which concentration is in one to one correspondence with the concentration of the body analyte in the body fluid due to the diffusional equilibrium established in the microdialysis needle, may be analyzed in a number of ways. In the above example where the body analyte is glucose and the enzyme is glucose oxidase, the pH change of the perfusate and enzyme solution mixture due to the creation of the gluconic acid may be measured, but this is difficult due to body fluid buffers that also enter the perfusate solution while it is in the microdialysis needle. Or, the change in oxygen concentration in the perfusate can be measured. Preferably, the hydrogen peroxide created during the reaction of the analyte and the enzyme is assayed electrochemically. Accordingly, a working electrode and a reference electrode are placed in the analysis chamber, preferably one each on the two large facing surfaces of the analysis chamber. Alternatively, an auxiliary third electrode to protect the reference electrode from degradation may be also placed in the analysis chamber. In this preferred embodiment, at the working electrode, hydrogen peroxide is reduced to create two electrons for each molecule of hydrogen peroxide. The working electrode may preferably be platinum or gold, and the reference electrode is preferably silver/silver chloride.

To perform the analysis in the analysis chamber, at some point in time after the perfusate has begun to flow through the system, the analysis chamber has been filled with the mixture of the body analyte laden perfusate and enzyme solution. At a selected time after the analysis chamber has been so filled, all fluid flow in the system is stopped and an appropriate voltage is placed on the electrode to cause the desired reaction of the hydrogen peroxide. Preferably, the voltage between the working electrode and the reference electrode is between 0.1 volt and 1.0 volt. This reaction is continued until virtually all of the hydrogen peroxide from the glucose/glucose oxidase reaction of the above example in the analysis chamber is reacted. In a preferred embodiment of the invention, the analysis chamber is 1 mm square by 20 microns high. At a flow rate of 2 nanoliters per second, the analysis chamber is filled with a fresh volume of perfusate and enzyme mixture in ten seconds. In this preferred embodiment, the time for diffusion from one side of the analysis chamber to the other for the hydrogen peroxide is less than 0.5 seconds. Hence, in a reaction time of 15 seconds, virtually all of the hydrogen peroxide will have been consumed. In this analysis scheme, instead of using the rate at which a reaction occurs, which leads to a current, the entirety of the reaction is measured, which leads to an electronic charge, measured in coulombs (electronic charge is the integral of current over time). Thus the measured electronic charge is a direct measure of the quantity of body analyte that was captured during the time the amount of perfusate in the analysis chamber was in the microdialysis needle. And since diffusional equilibrium was achieved in the microdialysis needle, this measured electronic charge is a direct measure of the concentration of the body analyte in the body tissue.

As is well known, enzyme catalyzed reactions, such as the glucose/glucose oxidase reaction in the presence of oxygen, are unstable, temperature dependent, and subject to losses in sensitivity, that is, the amount of current generated per unit concentration of substrate. While much of the latter problem is avoided by allowing the reaction in the analysis chamber to go to completion, all of these problems are avoided in the present invention by providing a calibration step that may be performed as frequently as desired, up to a one to one alternation with the perfusate.

To perform a calibration, perfusate flow is stopped, and flow from the calibration fluid reservoir is started with the calibration fluid entering the microdialysis chip at entry 4. Any flow in the direction of the outlet of the microdialysis needle is blocked by check valve 5. Since there is no perfusate flow, the calibration fluid and the enzyme solution meet at junction 15 and proceed to the mixing channel 6. The ensuing operation is similar to that for the perfusate. The enzyme and the calibration fluid mix by diffusion in the mixing channel, and this mixture flows into the analysis chamber. After an appropriate time, all fluid flow is stopped, and electrochemical analysis of the hydrogen peroxide reaction product is preferably measured. Since the concentration of the body analyte in the calibration fluid is known, the sensitivity of the assay may be directly calculated. This sensitivity may be used for subsequent analyses of the perfusate.

In a preferred embodiment of the invention, the sequence of perfusate and calibration analysis can be achieved as shown in FIG. 4. A tube pinching bar 35 is provided with perfusate flow tube 31, enzyme solution flow tube 32, and calibration flow tube 33 shown above pinch tube bar 35. Flow sequencing bar 34 is caused to move over the three flow tubes, stopping flow by closing the lumen of selected tubes as shown. FIG. 4 shows a sequence of calibration that alternates an assay of the perfusate from the microdialysis needle with an assay of the calibration fluid. In the uppermost drawing, all flow is stopped, in this case, so that an assay of the perfusate may be conducted. Then sequencing bar 34 is moved to the right, opening enzyme solution flow tube 32 and calibration flow tube 33. These fluids flow into the microdialysis chip as described above to position a reacted calibration fluid in the analysis chamber. At an appropriate time, sequencing bar is moved back to the left as shown in the third drawing from the top to stop all flow, allowing the assay of the reacted calibration fluid. Again, at an appropriate time, the sequencing bar is moved to the left as shown in the bottom drawing in FIG. 4, allowing flow of the perfusate and the enzyme solution. The perfusate passes through the microdialysis needle, mixes and reacts with the enzyme solution and moves to the analysis chamber as described above. Again, at an appropriate time, sequencing bar 34 is moved to the right, achieving again the position as shown in the top drawing of FIG. 4. While the flow is stopped, the analysis of the reacted perfusate in the analysis chamber is conducted. In this preferred embodiment, analysis of the perfusate and the calibration fluid alternate. Also, in this preferred embodiment, the motion of sequencing bar 34 is cyclic, that is the action of the bar may be achieved by using a single solenoid advancing a cam, each position of sequencing bar 34 achieved by rotating the cam 90 degrees.

The timing that can be achieved in this preferred embodiment may be as follows, but many other attractive timing sequences may be achieved. For the dimensions and flow rates of the system given above, the elapsed time from the time the perfusate begins to fill the microdialysis needle to the time this fluid finishes filling the analysis chamber is just under 40 seconds. Thus, the system of the preferred embodiment could operate as follows. First, run perfusate and enzyme solution for 45 seconds. Stop the flow for 15 seconds, allowing complete analysis and measurement in the analysis chamber. After 90 seconds, run the calibration fluid and enzyme solution for 45 seconds. Stop the flow for 15 seconds, allowing complete analysis and measurement of the calibration fluid. After 90 seconds, run the perfusate and enzyme solution for 45 seconds again. Stop the flow for 15 seconds, allowing for complete analysis and measurement of the perfusate in the analysis chamber. After 90 seconds, run the calibration fluid and enzyme solution for 45 seconds, and so on, continually repeating this sequence. This sequence of operation provides a new and accurate measurement of the perfusate every 5 minutes, checked before and after with an assay of the calibration fluid.

The total volume of fluid required to operate this system for 24 hours is minimal. Just 13 microliters of perfusate is needed. For the calibration fluid, again only 13 microliters of fluid is needed. For the enzyme solution, assuming a 1:1 mixture with the perfusate or the calibration fluid, only 26 microliters of fluid is needed. The total fluid requirement for a day's operation is just over 50 microliters. With such a minimal fluid requirement, a multiday system can be envisioned. Clearly, these minimal fluid requirements are a great improvement over the fluid requirements of Pfeiffer in U.S. Pat. No. 5,640,954 and an important improvement over the fluid requirements of Korf in U.S. Pat. No. 6,013,029.

While specific embodiments of the invention have been described in detail, it will be appreciated by those skilled in the art that various modifications and alternatives to those details could be developed in light of the teaching of the disclosure. Accordingly, the particular embodiment described in detail is meant to be illustrative and not limiting as to the scope of the invention, which is to be given the full breadth of the appended claims and any and all equivalents thereof.

I claim:

1. A body analyte monitoring device comprising
a) a microdialysis needle with an inlet port and an outlet port
b) a reservoir containing a perfusate in fluid communication with said inlet port of said microdialysis needle
c) the outlet port of said microdialysis needle in fluid communication with an analysis compartment wherein said body analyte acquired by said microdialysis needle may be assayed
c) pumping means to move said perfusate from said reservoir into said inlet port of said microdialysis needle, through said microdialysis needle, out of said outlet port of said microdialysis needle, and into said analysis compartment
d) a reservoir containing a body analyte calibration fluid in fluid communication with said analysis compartment
e) pumping means to move said calibration fluid from said calibration fluid containing reservoir into said analysis compartment
f) a reservoir containing a reagent solution in fluid communication with said analysis compartment
g) pumping means to move said reagent solution from said reagent solution reservoir into said analysis compartment
h) fluid sequencing means whereby the fluid moving into said analysis compartment may be selected as either said perfusate from said outlet port of said microdialysis needle mixed with said reagent solution or said calibration fluid mixed with said reagent solution.

2. The device of the claim 1 wherein said microdialysis needle is placed in contact with interstitial fluid of an animal.

3. The device of claim 2 wherein said microdialysis needle comprises a semipermeable membrane along one face of said microdialysis needle such that said semipermeable membrane is interposed between said interstitial fluid of an animal and the lumen of said microdialysis needle such that said body analyte passes from said interstitial fluid into said lumen.

4. The device of claim 3 wherein said semipermeable membrane is comprised of permeable polysilicon.

5. The device of claim 2 wherein the body analyte monitoring device is worn on the body in contact with the skin.

6. The device of claim 5 wherein body contact is maintained with a skin adhesive or a strap.

7. The device of claim 1 wherein said pumping means is a positive displacement pump or a pressurized reservoir or a piezoelectric pump.

8. The device of claim 1 wherein said microdialysis needle is made of silicon and in part fabricated using a silicon etch process.

9. The device of claim 1 wherein the cross-sectional area of the lumen of the microdialysis needle is less than 5000 square microns

10. The device of claim 1 wherein the width to height ratio of the cross-sectional area of the lumen of the microdialysis needle is larger than 1.5:1

11. The device of claim 2 wherein said body analyte is glucose.

12. The device of claim 11 in combination with a means for administering insulin.

13. The device of claim 12 wherein measurements of glucose are used to control the delivery of insulin from said means for administering insulin to said animal.

14. The device of claim 1 wherein the assay conducted in said analysis chamber is a measurement of an optical property of either the body analyte laden perfusate or the calibration fluid after mixing and reaction with said reagent solution.

15. The device of claim 1 wherein the assay conducted in said analysis chamber is a measurement of the viscosity of the body analyte laden perfusate or the calibration fluid after mixing and reaction with said reagent solution.

16. The device of claim 1 wherein the assay conducted in said analysis chamber is an electrochemical measurement of the body analyte laden perfusate or the calibration fluid after mixing and reaction with said reagent solution.

17. A periodic body analyte monitoring device comprising
a) a microdialysis needle with an inlet port and an outlet port
b) a reservoir containing a perfusate in fluid communication with said inlet port of said microdialysis needle
c) the outlet port of said microdialysis needle in fluid communication with an analysis compartment wherein said body analyte may be assayed
c) pumping means to move said perfusate from said perfusate reservoir into said inlet port of said microdialysis needle, through said microdialysis needle, and out of said outlet port of said microdialysis needle, and into said analysis compartment
d) a reservoir containing a solution of an enzyme specific for said body analyte in fluid communication with said analysis compartment
e) pumping means to move said enzyme solution from said enzyme solution reservoir into said analysis compartment
f) a reservoir containing a body analyte calibration fluid in communication with said analysis compartment
g) pumping means to move said calibration fluid from said calibration fluid containing reservoir into said analysis compartment
h) fluid sequencing means whereby the fluid moving into said analysis compartment can be selected as either said perfusate mixed with said enzyme solution or said calibration fluid mixed with said enzyme solution.

18. The device of the claim 17 wherein said microdialysis needle is placed in contact with interstitial fluid of an animal.

19. The device of claim 18 wherein said microdialysis needle comprises a semipermeable membrane along one face of said microdialysis needle such that said semipermeable membrane is interposed between said interstitial fluid of an animal and the lumen of said microdialysis needle such that said body analyte passes from said interstitial fluid into said lumen.

20. The device of claim 21 wherein said semipermeable membrane is comprised of permeable polysilicon.

21. The device of claim 17 wherein the periodic body analyte monitoring device is worn on the body in contact with the skin

22. The device of claim 21 wherein body contact is maintained with a skin adhesive or a strap.

23. The device of claim 17 wherein said pumping means is a positive displacement pump or a pressurized reservoir or a piezoelectric pump.

24. The device of claim 17 wherein said microdialysis needle is made of silicon and in part fabricated using a silicon etch process.

25. The device of claim 17 wherein the cross-sectional area of the lumen of said microdialysis needle is less than 5000 square microns

26. The device of claim 17 wherein the width to height ratio of the cross-sectional area of the lumen of said microdialysis needle is larger than 1.5:1

27. The device of claim 18 wherein said body analyte is glucose

28. The device of claim 27 in combination with a means for administering insulin.

29. The device of claim 28 wherein measurements of glucose are used to control the delivery of insulin from said means for administering insulin to said animal.

30. A method of calibrating a body analyte monitoring microdialysis device comprising alternating the flow of a body analyte laden perfusate mixed with an appropriate reagent and a body analyte containing calibration fluid mixed with said appropriate reagent into an analysis compartment wherein a result of a reaction between said appropriate reagent and the body analyte in said body analyte containing perfusate or calibration fluid is measured.

31. The method of claim 30 wherein at least one portion of said body analyte containing calibration fluid mixed with said appropriate reagent enters said analysis compartment before a portion of said body analyte containing perfusate mixed with said appropriate reagent enters said analysis compartment.
